(19) United States
(12) Patent Application Publication
(10) Pub. No.: US 2004/0089058 A1
(43) Pub. Date: May 13, 2004

(54) Sensor for detecting the presence of moisture

(76) Inventors: Peter Hillebrand De Haan, Delft (NL); Ronald Henri Marie Van De Leur, Best (NL)

Correspondence Address: Muserlian, Lucas and Mercanti, LLP, 475 Park Avenue South, New York, NY, 10016, US

(21) Appl. No.: 10/701,840

(22) Filed: Nov. 4, 2003

Related U.S. Application Data
(62) Division of application No. 10/070,576, filed on Aug. 27, 2002.
Application No. 10/070,576 is a 371 of international application No. PCT/NL00/00639, filed on Sep. 11, 2000.

(30) Foreign Application Priority Data
Sep. 9, 1999 (NL) 1013012

(51) Int. Cl. G01N 27/22; G01N 27/00; A61F 13/42

(52) U.S. Cl. 73/73; 604/385.01; 324/664

(57) ABSTRACT

The invention relates to a sensor for detecting the presence of moisture. The sensor comprise an electric circuit and IPs. Electric properties of the ICPs are dependent on the amount of moisture with which the come into contact. The electric circuit is arranged for detecting a change of the electric properties of the ICPs for detecting moisture. The ICPs form part of a capacity, the electric circuit being arranged for detecting a change of the capacity for detecting moisture.

The invention relates to a moisture sensor for detecting the presence of moisture comprising an electric circuit and ICPs (intrinsic conduction polymers), electric properties of the ICPs being dependent on the amount of moisture with which they come into contact and the electric circuit being arranged for detecting a change of the electric properties of the ICPs for detecting moisture.

Such moisture sensor is known per se. In the known moisture sensor, the ICPs form part of the electric circuit, the electric circuit being arranged for detecting a change of the resistance of the ICPs on the basis of a change in the presence of the moisture.

A drawback of the known device is that the detection of moisture cannot be performed particularly accurately. In particular when the moisture sensor is used in a baby diaper, incontinence diaper, mattress and/or incubator for detecting bodily fluid, such as urine, detection of a delivery of relatively small amounts of bodily fluid often proves to be impossible.

The object of the invention is to provide a moisture sensor which enables detection of the presence of relatively small amounts of moisture. According to a first further elaboration of the invention, it applies that the ICPs form part of a capacity, the electric circuit being arranged for detecting a change of the capacity for detecting the moisture. Hence, according to this elaboration, it applies that instead of a resistance measurement, a capacitive measurement is performed.

More in particular, it applies that on a first side of a substrate, there is provided a layer comprising the ICPs and that on a second side of the substrate opposite the first side, electrodes are provided which together with the layer form part of the capacity.

However, it is also possible that on a first side of a substrate, there is provided a layer comprising the ICPs, while at least one first electrode is provided on a second side of the substrate opposite the first side and at least one second electrode is provided on the layer, such that the layer lies at least partly between the first and the second electrode, the first and second electrodes together with the ICPs forming part of the capacity. According to a further elaboration of this variant, it applies that on the first side of the substrate, there are also provided conductive paths which together with the capacity form an LC circuit of the electric circuit.

For performing a capacitive measurement, according to a further elaboration of this aspect of the invention, it is also possible that on a first side of a substrate, there is provided a layer comprising the ICPs, while at least one electrically conductive path comprising windings is provided on a second side of the substrate opposite the first side, the electrically conductive path forming a coil of the electric circuit and, together with the layer, the capacity.

According to an alternative embodiment of the invention, which also meets the problems outlined, it applies that the electric circuit is provided with a transponder incorporated into a casing which is at least partially manufactured from the ICPs. The casing functions as a Faraday cage which, depending on the presence of moisture, shields the transponder to a more or lesser degree from a space located outside the casing. Hence, when, by means of the transceiver, an interrogation field is generated into which the moisture sensor is introduced, the magnitude of a response from the transponder received by the transceiver will depend on the degree to which the transponder is shielded by the casing, i.e. depend on the amount of moisture present in the proximity of the moisture sensor. Here, the transceiver and transponder may operate according to the principle of the absorption or transmission systems known per se.

According to an alternative variant of the invention meeting the problems stated, it applies that the moisture sensor comprises a current-conductive fabric comprising ICPs.

In particular, it applies that the moisture sensor is provided with at least one pair of electrodes forming part of the electric circuit and connected to the fabric at different locations. Possibly, the moisture sensor is provided with at least two pairs of electrodes.

According to this particular embodiment, by determining, by means of the electric circuit, the resistance between the electrodes of each pair, it is possible to estimate the position where the moisture is located on the fabric. A crossbearing is performed, as it were.

The invention will hereinafter be specified with reference to the accompanying drawings. In these drawings:

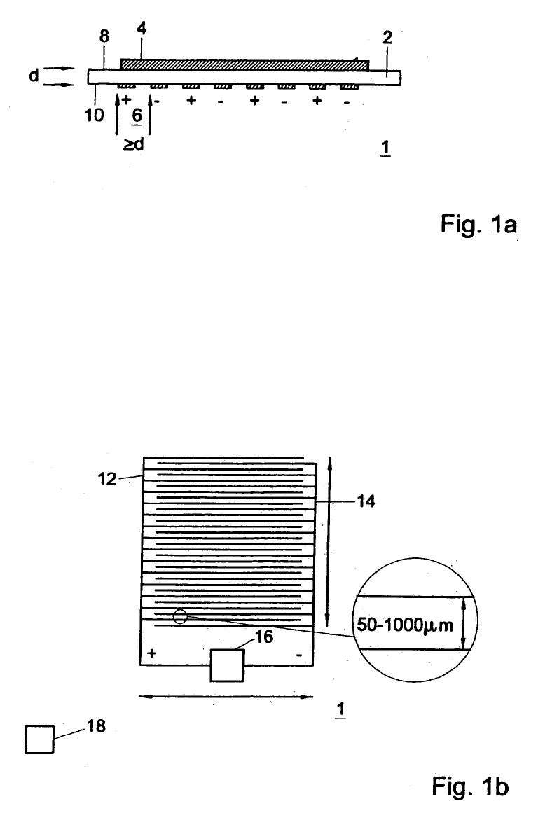

FIG. 1a is a side elevation of a first embodiment of a moisture sensor according to the invention;

FIG. 1b is a top plan view of the moisture sensor according to FIG. 1a;

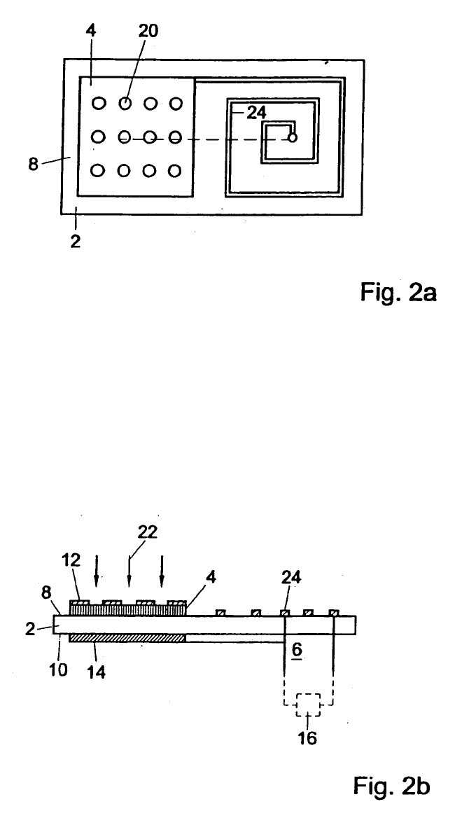

FIG. 2a is a top plan view of a second embodiment of a moisture sensor according to the invention;

FIG. 2b is a side elevation of the moisture sensor according to FIG. 2a;

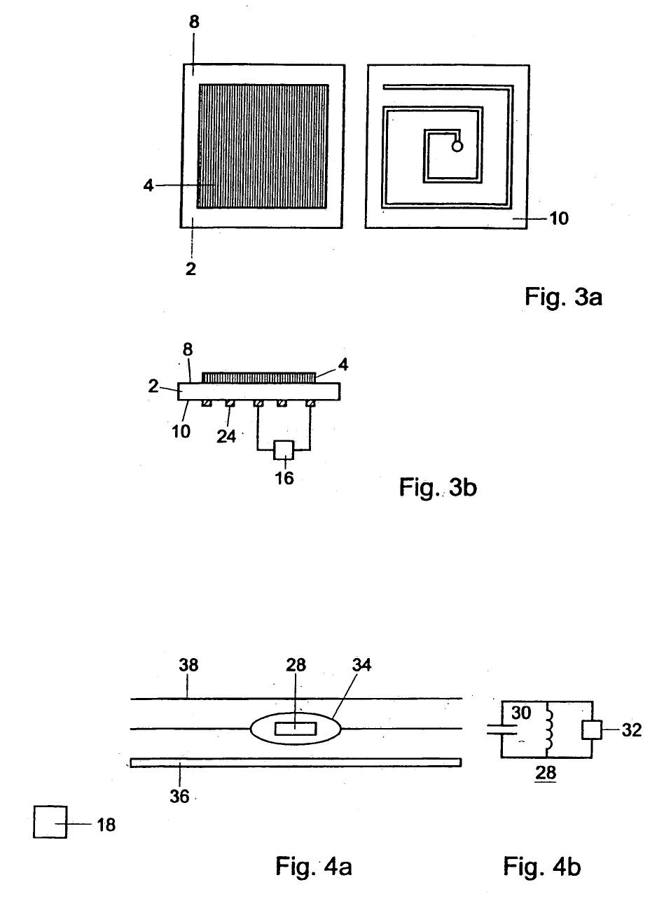

FIG. 3a is a top plan view of a third embodiment of a moisture sensor according to the invention;

FIG. 3b is a side elevation of the moisture sensor according to FIG. 3a;

FIG. 4a is a side elevation of a fourth embodiment of a moisture sensor according to the invention, incorporated into a diaper;

FIG. 4b shows a resonant circuit of the moisture sensor according to FIG. 4a;

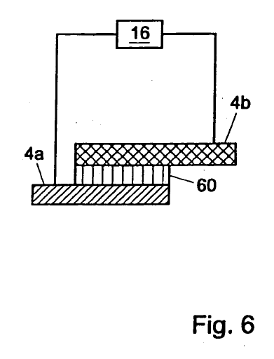

FIG. 6a is a top plan view of a fifth and sixth embodiment of a moisture sensor according to the invention;

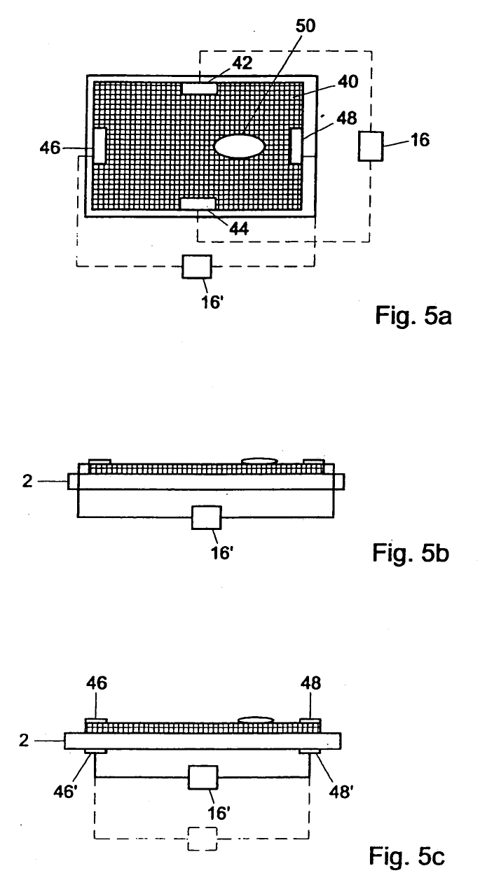

FIG. 5b is a side elevation of the fifth embodiment of the moisture sensor according to FIG. 5a;

FIG. 5c is a side elevation of the sixth embodiment of the moisture sensor according to FIG. 5a; and

FIG. 6 is a side elevation of a seventh embodiment according to the invention.

In FIGS. 1a and 1b, reference numeral 1 designates a moisture sensor according to the invention. The moisture sensor comprises a substrate 2. This substrate can, for instance, consist of one of the inner layers of a diaper, a mattress, an incubator and the like, depending on the use of the moisture sensor. Hence, the substrate 2 can consist of, for instance, a plastic layer, a textile layer, such as a nonwoven, a paper layer, etc. Provided on the substrate 2 is an intrinsic conduction polymer 4 in the form of a layer 4. Due to their chemical structure, intrinsic conduction polymers exhibit semiconducting properties. The properties of conductive polymers depend on the environment.

ICPs are known per se. Thus, in the late eighties, the US textile company Milliken developed a technique for applying a thin layer of polypyrrole to the separate fabrics of textile, such as, for instance, polyester (trademark: Contex). In addition, other types of ICPs are known, such as polythiophene (and derivatives), polyaniline and the like.

According to the invention, use is made of the fact that electric properties of ICPs depend on the amount of moisture with which the ICPs come into contact. The moisture sensor further comprises an electric circuit 6 arranged for detecting a change of the electric properties of the ICPs for detecting moisture.

In this example, the layer of ICPs 4 is applied to a first side 8 of the substrate 2. On a second side 10 opposite the first side 8 of the substrate 2, electrodes 12, 14 are provided. In this example, the electrodes 12, 14 are designed as a comb capacitor known per se. The electric circuit 6 comprises a measuring unit 6 connected to the electrodes 12, 14 of the comb capacitor 16. Together with the layer of ICPs 4, the comb capacitor 16 forms a capacity that can be measured in a manner known per se by means of the measuring device 16. Because the electric properties (resistance, electric susceptibility) of the intrinsic conduction polymer 4 depend on the amount of moisture with which the layer of ICPs comes into contact, the moisture can thus be measured by means of the measuring device 16. The capacity (and the corresponding loss factor) of the ICP layer can be measured by generating, by means of the measuring device 16, signals having different frequencies and determining the amplitudes of these signals. A change in the capacity due to the presence of moisture can thus be detected.

The measuring device 16 can hence comprise, for instance, an AC generator. It is also possible that the measuring device 16 is of such design that the capacity determined by means of the measuring device 16 can be supplied wirelessly to a reading device 18 (FIG. 1b).

It is also possible that the capacity formed by the electrodes 12, 14 and the layer of ICPs 4 forms, together with the measuring device 16, a resonant circuit known per se, as used for anti-theft labels. This resonant circuit may then further comprise a chip including an identification code. In this manner, by means of the reading device 18, which in this example generates an interrogation field, the amount of moisture detected by the moisture sensor 1 can be read out together with an identification code of the moisture sensor. Such system can advantageously be used in, for instance, a hospital, where the mattresses of hospital beds can each be provided with a moisture sensor 1. Because each moisture sensor has its own identification code, it is possible that by means of the reading device 18, it is not only established that one of the mattresses has become wet, but also which mattress has become wet.

A second possible embodiment of a moisture sensor according to the invention will be discussed with reference to FIGS. 2a and 2b. Parts corresponding to FIG. 1 have been provided with identical reference numerals. In this embodiment, a layer of ICPs 4 has again been applied to the substrate 2. On the layer 4, an electrode 12 is provided. On the second side 10, there is provided a plate-shaped electrode 14. The electrode 12 is provided on the layer 4 in such a manner, that the layer 4 lies at least partly in between the electrodes 12, 14. The electrode 12 is plate-shaped as well. However, in the electrode 12, a number of openings 26 are provided so that the layer of ICPs 4 is accessible for moisture 22 located on the first side 8 of the substrate. Together with the layer of ICPs 4, the electrodes 12, 14 again form part of a capacity. Further, it applies that on the first side 8 of the substrate, conductive paths 24 are provided, manufactured from, for instance, copper. The conductive paths 24 form, together with the above-mentioned capacity 4, 2, 14, an LC circuit forming part of the electric circuit 6. The resonance frequency of the LC circuit formed will depend on the electric properties of the layer of ICPs and, accordingly, of the amount of moisture in the environment of the moisture sensor. The resonance frequency can again be determined in a manner known per se by means of the measuring device 16. For this purpose, the measuring device 16 may be provided with an AC generator by means of which a frequency-varying AC signal can be supplied to the LC circuit for determining the resonance frequency. However, as discussed in respect of FIG. 1a, it is also possible that the LC circuit and the measuring device 16 form a part of a resonant circuit that can be read out by means of the read-out device 18 when it is introduced into an interrogation field generated by the read-out device 18. Then, the measuring device 16 is, for instance, a simple short circuit. In that case, an interrogation field is generated by the measuring device 18, by means of which the resonance frequency of the LC circuit is determined. In this example, the measuring device 16 can then also be replaced by a chip 16 in which, again, an identification code of the sensor 1 is stored. When the LC circuit is introduced into the interrogation field, this chip 16 can modulate the current through the LC circuit with the identification code. This modulated current can be detected by the read-out unit 18.

In the exemplary embodiment according to FIGS. 3a and 3b, parts corresponding to FIG. 1 are again provided with the same reference numerals. Again, it applies that on the first side 8 of the substrate, the layer 4 comprising the ICPs is provided. Further, at least one electrically conductive path 24 comprising windings is provided on the second side 10 of the substrate. This electrically conductive path forms a coil. The ends of the coil 24 are connected to the measuring device 16. Because the coil 24 and the substrate 4 are disposed on opposite sides of the substrate 2, they also form, in combination, a capacity. By means of the measuring device 16, the resonance frequency can again be determined of the LC circuit formed by the coil and the layer of ICPs, as discussed in respect of FIG. 2.

FIG. 4a shows a fourth possible embodiment of a moisture sensor according to the invention. The moisture sensor comprises a transponder known per se, of the type which is, for instance, used for anti-theft labels. The transponder 28 can, for instance, comprise a resonant LC circuit 30 (see FIG. 4b) and a chip 32 coupled to the resonant circuit 30. When the transponder 28 is introduced into an electromagnetic interrogation field generated by means of a read-out unit 28, the transponder 28 will respond thereto when the frequency of the interrogation field corresponds to the resonance frequency of the LC circuit 30. The system formed by the read-out unit 18 and the transponder 28 can function according to the transmission principle known per se, as well as according to the absorption principle known per se. According to the invention, the transponder 28 is included in a casing 34 which is at least partially formed by ICPs. This casing can, for instance, consist of a foil manufactured from ICPs or a foil to which a coating of ICPs has been applied. Because the resistance of the ICPs of the casing 34 is low, the casing will act as a Faraday cage and shield the transponder 28 from the interrogation field generated by means of the read-out device 18. The read-out device 8 will then detect no transponder 28. However, the properties of the ICPs have been selected such that when the ICP comes into contact with water (for instance urine), the resistance increases. Accordingly, the shielding of the transponder 28 decreases, enabling the read-out unit 18 to detect the transponder 28. Hence, the moisture located in the environment of the casing 34 is thus detected. The casing 34 can, for instance, be incorporated into a diaper, as shown in FIG. 4a, with the layer 36 forming an outer layer (cover stock) of the diaper and the layer 38 forming an inner layer of the diaper. Also, in this manner, by means of the reading device 18, the magnitude of the, resistance of the ICPs can be determined on the basis of the strength of the signal received. This magnitude of the resistance is then a measure for the moistness at the sensor.

FIGS. 5a and 5b show a fifth embodiment of a moisture sensor according to the invention. The moisture sensor is provided with a current-conductive fabric 40 comprising ICPs. This type of fabric is used, inter alia, for manufacturing antistatic clothing. The DC resistance of the fabric is, for instance, in the range of from 20 Ω to 20,000 kΩ. The moisture sensor further comprises at least one pair and in this example at least two pairs of electrodes 42, 44; 46, 48, connected to the fabric at different locations. In this example, the electrodes 42, 44 are placed adjacent opposite longitudinal edges of the fabric. Further, the electrodes 46, 48 are placed on two other, opposite longitudinal edges of the fabric. However, the precise position of the electrodes is not relevant. By means of a measuring device 16, the impedance between the relevant electrodes 42, 44 is measured. This impedance is measured at a suitable frequency in the frequency range of, for instance, 20 Hz to 1 MHz. When moisture gets on the fabric, the impedance between the electrodes 42, 44 will change through short-circuiting between the conductive wires in the fabric and due to the intrinsic properties of the moisture (such as electric conductivity and high dielectric constant). In this manner, the moisture 50 can be detected. Entirely analogously, by means of the electrodes 46, 48 and a second measuring device 16′, the moisture 50 can be detected as well. The impedance change measured by means of the electrodes 46, 48 will depend on the position of the moisture on the fabric 40. This also applies to the impedance change effected by the moisture 50 and measured by means of the electrodes 42, 44 and the measuring device 16. By processing the impedance changes determined by means of the first measuring device 16 and the second measuring device 16′ in combination, information can be obtained about the position of the moisture 50 on the fabric 40.

The relevant fabric 40 can be provided in, for instance, a diaper in a suitable position, for instance directly below the first moisture-permeable layer which comes into contact with the skin, centrally in the urine-repellent layer or below the absorbing layer directly above the cover layer. The connections of the electrodes can be passed outside through the cover layer or passed through the inside of the diaper to the edge. The first measuring device 16 and the second measuring device 16′ can then be measuring devices disposed outside the diaper. Of course, it is also possible that the first measuring device 16 comprises an LC circuit forming, together with the electrodes 42, 44 and the fabric 40, a circuitry, known per se, of an anti-theft label. Thus, in an entirely analogous manner, the electrodes 46, 48, the fabric 40 and the second measuring device 16′ can form a second transponder of an anti-theft circuitry. In this manner, two transponders are formed which, when having mutually different resonance frequencies, can be separately detected by means of the read-out device 18 which can then generate at least two interrogation fields. The first interrogation field corresponds to the resonance frequency of the transponder 16, 40, 42, 44, while the frequency of the second interrogation field corresponds to the resonance frequency of the transponder 16′, 40, 46, 48. The impedance formed by the electrodes 42, 44 and the fabric 40 can be incorporated into the LC circuit of the first transponder in such a manner that the Q factor of the transponder drops when the impedance of the fabric 40 increases. However, it is also possible that the relevant impedance is incorporated into the LC circuit such that the Q factor rises when the relevant impedance increases. This applies entirely analogously to the transponder formed by the measuring device 16′, electrodes 46, 48 and the fabric 40. Again, depending on the determination of a change of the Q factors of the first and second transponders, information can be obtained about the position of the moisture 50 on the fabric.

FIG. 5c father shows that the electrodes 46, 48 can be connected to the read-out unit 16′ via a capacitive coupling. To this end, opposite the electrode 46, an electrode 46′ is arranged, while opposite the electrode 48, an electrode 48′ is arranged. Electrodes 46′, 48′ are then connected to the read-out device 16′. If, for instance, the substrate 2 is formed by the outer layer of a diaper, the device 16′ can readily be connected to the electrodes 46′ and 48′, on the condition that the device 16′ generates a sufficiently high frequency for determining the impedance between the electrodes 46 and 48. In this respect, one may, for instance, think of a frequency greater than 100 kHz. Entirely analogously, the device 16 can determine the impedance between the electrodes 42 and 44 by means of electrodes 42′ and 44′.

In the embodiment according to FIG. 6, use is made of the difference in electrochemical potential of two different ICPs. A sensor is formed by separating two layers 4a, 4b of different ICPs (for instance polypyrrole and polyaniline or polypyrrole and polythiophene) or a layer of ICP with a layer of metal (for instance aluminum) from each other by a dielectric 60 capable of absorbing moisture (for instance tissue paper). As measuring device, a voltmeter 16 is connected to the two different ICPs or to the ICP and metal layer. If the sensor is dry, no voltage difference will be measured between the two layers, because the dielectric is not conductive and hence no charge exchange takes place between the two different current-conductive layers. If the sensor is wet, the dielectric becomes (slightly) conductive through the absorbed water (or urine), so that the voltmeter can indicate the difference in the electrochemical potential of the two materials. It has been found that the potential difference (open terminal voltage) in the case of a layer of polypyrrole and aluminum is about 700-800 mV and the potential difference in the case of a layer of polypyrrole and polyaniline is about 100 mV. The potential difference at longitudinal electric load (so-called open terminal voltage) is independent of the amount of moisture supplied, provided that the conductivity is sufficient. The short-circuit current can in fact be dependent on the amount of moisture supplied. Further, in the embodiment according to FIGS. 5a-5c, it is also possible that the detection is not based on changes in the intrinsic electric properties of the ICP, but on the changes of the electric response due to the presence of the absorbed water in the fabric or water on the fabric. The dielectric constant of water is very high and the conductivity is high, so that a change in the dielectric response can certainly occur. In this case, use is made of the current-conducting fabric comprising ICP as electrode. Such variants are each understood to fall within the framework of the invention.

1. A moisture sensor for detecting the presence of moisture, comprising an electric circuit and ICPs (intrinsic conduction polymers), electric properties of the ICPs being dependent on the amount of moisture with which they come into contact and the electric circuit, being arranged for detecting a change of the electric properties of the ICPs for detecting moisture, characterized in that the ICPs form part of a capacity, the electric circuit being arranged for detecting a change of the capacity for detecting the moisture.

2. A moisture sensor according to claim 1, characterized in that on a first side of a substrate, there is provided a layer comprising the ICPs and that on a second side of the substrate opposite the first side, electrodes are provided which together with the layer form part of the capacity.

3. A moisture sensor according to claim 2, characterized in that the electrodes are designed as a comb capacitor.

4. A moisture sensor according to claim 1, characterized in that on a first side of a substrate, there is provided a layer comprising the ICPs, while at least one first electrode is provided on a second side of the substrate opposite the first side and at least one second electrode is provided on the layer, such that the layer lies at least partly between the first and the second electrode, the first and second electrodes together with the ICPs forming part of the capacity.

5. A moisture sensor according to claim 4, characterized in that on the first side of the substrate, there are also provided conductive paths which together with the capacity form an LC circuit of the electric circuit.

6. A moisture sensor according to claim 1, characterized in that on a first side of a substrate, there is provided a layer comprising the ICPs, while at least one electrically conductive path comprising windings is provided on a second side of the substrate opposite the first side, the electrically conductive path forming a coil of the electric circuit and, together with the layer, the capacity.

7. A moisture sensor according to any one of the preceding claims, characterized in that the electric circuit comprises a transponder for obtaining wirelessly information about the detected presence of the moisture.

8. A moisture sensor according to claim 7, characterized in that the transponder comprises a resonant circuit comprising the capacity.

9. A moisture sensor for detecting the presence of moisture, comprising an electric circuit and ICPs (intrinsic conduction polymers), electric properties of the ICPs being dependent on the amount of moisture with which they come into contact and the electric circuit being arranged for detecting a change of the electric properties of the ICPs for detecting the moisture, characterized in that the electric circuit comprises a transponder incorporated into a casing at least partially manufactured from the ICPs.

10. A moisture sensor according to claim 9, characterized in that the properties of the ICPs are such that the resistance of the ICPs increases when they come into contact with the moisture.

11. A moisture sensor according to claim 1, characterized in that the moisture sensor comprises a current-conductive fabric comprising ICPs, at least one pair of electrodes connected to a first side of the fabric and electrodes located on a second side of the fabric opposite the first side, connected to the first and the second pair of electrodes by means of a capacitive coupling and forming part of the electric circuit.

12. A moisture sensor for detecting the presence of moisture, comprising an electric circuit and ICPs (intrinsic conduction polymers), electric properties of the ICPs being dependent on the amount of moisture with which they come into contact and the electric circuit being arranged for detecting a change of the electric properties of the ICPs for detecting moisture, characterized in that the moisture sensor comprises a current-conductive fabric comprising the ICPs.

13. A moisture sensor according to claim 12, characterized in that the moisture sensor comprises at least one pair of electrodes forming part of the electric circuit and connected to the fabric at different locations.

14. A moisture sensor according to claim 13, characterized in that the at least one pair of electrodes is connected to a first side of the fabric and that the electric circuit further comprises electrodes disposed on a second side of the fabric opposite the first side and connected, by means of a capacitive coupling, to the at least one pair of electrodes.

15. A moisture sensor according to claim 14, characterized in that the moisture sensor comprises at least two pairs of electrodes.

16. A moisture sensor for detecting the presence of moisture, comprising an electric circuit and ICPs (intrinsic conduction polymers), electric properties of the ICPs being dependent on the amount of moisture with which they come into contact and the electric circuit being arranged for detecting a change of the electric properties of the ICPs for detecting the moisture, characterized in that the moisture sensor comprises at least two layers, each layer comprising ICPs and a dielectric incorporated between the layers, the dielectric being of a type capable of absorbing moisture and the sensor being arranged for detecting, in use, the moisture on the basis of a voltage difference between the layers.

17. A diaper provided with a moisture sensor according to any one of the preceding claims.

18. A diaper provided with a moisture sensor according to any one of preceding claims 2-8, characterized in that the first side of the substrate faces an inner side of the diaper.
